(19) United States
(12) Patent Application Publication
(10) Pub. No.: US 2017/0251943 A1
(43) Pub. Date: Sep. 7, 2017

(54) Systems Level State Characteristics in Experimental Treatment of Disease

(71) Applicant: Per Fredrik PETERSSON, Malmö (SE)

(72) Inventors: Per Fredrik PETERSSON, Malmö (SE); Pär HALJE, Malmö (SE)

(21) Appl. No.: 15/506,397

(22) PCT Filed: Aug. 26, 2015
(86) PCT No.: PCT/EP2015/069555

§ 371 Date: Feb. 24, 2017

(30) Foreign Application Priority Data
Aug. 26, 2014 (SE) 1400404-8

(51) Int. Cl. A61B 5/04; A61B 5/00; A61B 5/0484

(52) U.S. Cl. CPC A61B 5/04017; A61B 5/0484; A61B 5/4082; A61B 5/4088; A61B 5/725; A61B 5/7257; A61B 5/4064

(57) ABSTRACT

Methods and system for assessment of a state of the central nervous system (CNS) of a subject and/or differentiation between states of the CNS at a specific time point based on electrophysiological recordings of signals from at least two anatomical structures, wherein the signals are recorded from recording sites located in the anatomical structures, wherein the electrophysiological recordings comprise spatiotemporal fluctuations in the recorded extracellular potential, wherein the electrophysiological recordings are action potentials and/or local field potentials (LFPs) in the anatomical structures and represent the state of the CNS, a s well as the use of the method for assessment of a state of the CNS and/or differentiation of at least two states of the CNS, as well as to the use of the method for evaluating the effect of a treatment of a condition or disease, wherein the condition or disease is neurological and/or psychiatric.

### TECHNICAL FIELD OF THE INVENTION

The invention relates to a method and a system for assessment and/or differentiation between states of the central nervous system (CNS) based on multi-structure electrophysiological recordings. In particular the invention relates to a method to characterize CNS states induced by disease or models of disease and/or experimental interventions aimed at modifying these states and/or treating disease.

### BACKGROUND ART

Diseases affecting the central nervous system have proven particularly hard to treat and have become a growing problem for society. A major reason behind the many challenges in developing new therapies is the fundamental lack of information on the underlying physiological processes in the healthy as well as the diseased nervous system.

The CNS is in essence an extremely complex network built of several interacting subsystems that communicate using signals acting on several different timescales. In electrophysiological recordings of CNS activity, electrodes are used to record voltage fluctuations either from within neurons or in the extracellular space in the close vicinity of neurons. These voltage fluctuations typically occur on a time scale of just below 1 ms up to a few seconds. The higher end of this frequency spectrum is typically analyzed to identify the signaling displayed by individual neurons in the form of action potentials generated by a neuron when its plasma membrane becomes sufficiently depolarized. These voltage fluctuations are transient in nature and spread along the plasma-membrane of the neuron. Both the timing and average rate of action potentials generated by each neuron can potentially carry information within and between connected regions of the CNS. Slower voltage fluctuations recorded in the extracellular space are, on the other hand, regarded to be generated primarily by synchronous activity in a larger number of neurons located close to the recording electrode (referred to as local field potentials; LFPs). These changes in electrical potential are assumed to arise as a result of the movement of charges stemming from synaptic activity that produces ion-currents spreading actively and passively through dendritic structures of the neurons, but also by the synchronous generation of action potentials in nearby cell groups. Thus, by measuring these electrophysiological signals the experimenter can gain access to parts of the information processing going on in both single cells and in populations of neurons in small volumes close to each recording electrode within the implanted anatomical structures of the CNS.

Through the development of large scale multichannel recording systems and improved methods for chronic implantation of extracellular electrodes several research groups have, during the last two decades, been able to provide more detailed descriptions of electrophysiological activity patterns associated with different brain processes governing motor behavior ( ), sensory processing) and, under certain conditions, characterizations of internal brain states (such as sleep states) and states created by interventions aimed at altering pathological activity patterns (Fanselow et al., 2000; Gervasoni et al., 2004; Fuentes et al., 2009; Santana et al 2014).

However, for these methods to become more widely applicable detailed information on how diseases, or interventions aimed at treating disease, affect CNS states need to be accessible in the small experimental animals (such as rats and mice) that are normally used in biomedical research.

### SUMMARY OF THE INVENTION

It is an objective of the present invention to provide an improvement of the above technique and prior art. More particularly, it is an objective of this invention to provide a method for assessing the state of the central nervous system in a subject.

According to a first aspect, these and other objects, and/or advantages that will be apparent from the following description of embodiments, are achieved, in full or at least in part, by a method for assessment of a state of the central nervous system (CNS) of a subject and/or differentiation between states of the central nervous system (CNS) of a subject at a specific time point based on electrophysiological recordings of signals from at least two anatomical structures. The signals are recorded from recording sites located in the anatomical structures. The electrophysiological recordings comprise spatiotemporal fluctuations in the recorded extracellular potential. The electrophysiological recordings are action potentials and/or local field potentials (LFPs) in the anatomical structures and represent the state of the CNS at said specific timepoint. The method comprises the steps of: a) amplifying recorded action potentials and/or local field potentials; b) digitizing recorded action potentials and/or local field potentials; c) reducing the influence of current dipoles located far away from the recording site to obtain signals that are decoupled from other anatomical structures. The method further comprises at least one of the following four steps d1) high-pass filtering of the signals and amplitude thresholding at a predefined value above noise background for the identification of action potentials; d2) obtaining at least one spike train from trains of action potentials generated from groups of neurons or individual cells; e1) obtaining power spectral densities (PSDs) or cross-spectral densities for the LFPs; e2) normalizing PSDs to noise background. The method further comprises the step of f) assessing the state of the CNS based on the recorded spatiotemporal fluctuations defining a location in a predefined state space; wherein the predefined state space is defined by a set of feature vectors.

In short, by using the information comprised in the recorded spatiotemporal fluctuations, the state of the CNS at a given time point may be localized in a state space. Depending on the location of the state in the state space, conclusions regarding the state of the CNS may be drawn. For example, the method according to the present invention may be used to discriminate between a healthy state and a diseased state. Importantly, the method according to the present invention may discriminate between states of the CNS which give rise to the same behaviour, but which differ in their electrophysiological activity patterns, in e.g. a subject who has received a treatment for a specific disease. This is important e.g. when evaluating the effect of new drugs or treatments of a disease. Even if the behaviour of the subject changes to the desired behaviour, the treatment may not have had the desired effect on the electrophysiological activity patterns of the brain, thus potentially leading to insufficient treatment effects and/or unwanted side effects.

Only spike trains may be used for the assessment of a state of the central nervous system (CNS) and/or for differentiation between states of the central nervous system (CNS). An advantage of using spike trains is that state dependent changes relating to the firing of specific cell groups can be included.

Only PSDs may be used for the assessment of a state of the central nervous system (CNS) and/or for differentiation between states of the central nervous system (CNS). An advantage of using PSDs is that synchronized oscillations in population activity in the vicinity of the measurement sites can be detected.

Only cross-spectral densities (CSDs) may be used for the assessment of a state of the central nervous system (CNS) and/or for differentiation between states of the central nervous system (CNS). An advantage of using CSDs is that synchronized and coherent activity in interconnected brain circuits can be detected.

A combination of two or more of spike trains, PSDs and CSDs may be used for the assessment of a state of the central nervous system (CNS) and/or for differentiation between states of the central nervous system (CNS). An advantage of using a combination of two or more of spike trains, PSDs and CSDs is that different aspects of the neuronal information processing can be assessed. Generally, a combination of these measures will result in a more detailed assessment of the neuronal information processing than any of the measures independently.

Step a) may be performed using any method for amplifying electrical signals. One example of such a method is the use of an operational amplifier.

Step b) may be performed using any method for digitizing electrical signals. One example of such a method is an A/D-conversion using at least 16 bits of sampling depth and at least 32 kHz sampling rate per channel.

Step c) may be performed by recording from multiple sites within the same anatomical structure and taking differential measures between sites. An advantage of performing this step is that the influence of current dipoles located far away from the recordings sites can be reduced.

Through steps d1) to d2) spike trains (time points of action potential events) are obtained. Such spike trains measure how information is processed and transmitted by single neurons or groups of neurons.

The high-pass filtering of the signals, step d1), may be performed with a cut-off frequency of 100-1000 Hz, preferably 300-700 Hz, most preferably 550-650 Hz. In one embodiment the cut-off frequency may be 600 Hz.

Step d2) may be performed using any method for obtaining spike trains. One example of such a method is amplitude thresholding followed by clustering based on the shape of the action potential waveform.

Through steps e1) and e2) PSDs are obtained. PSDs measure the synchronous activity of neuronal populations.

Step e1) may be performed using any method for obtaining power spectral densities (PSDs) or cross-spectral densities for the LFPs.

One example of a method for obtaining power spectral densities (PSDs) for the LFPs is the Fast Fourier Transform (FFT). Preferably, power spectral densities (PSDs) for the LFPs are obtained by the multitaper FFT method. An advantage of using PSDs is that they capture activity of neuronal populations that are not detectable by observing single units alone.

One example of a method for obtaining cross-spectral densities for the LFPs is the Fast Fourier Transform (FFT). Preferably, cross-spectral densities (CSDs) for the LFPs are obtained by the multitaper FFT method. An advantage of using cross-spectral densities is that it measures coherent activity between different anatomical structures between pairs of recording sites by taking the phase-information into account.

Step e2) may be performed using any method for normalizing PSDs to noise background. Preferably, this step is performed by estimating the noise background S(f), and taking log(PSD(f)/S(f)). An advantage of normalizing PSDs to noise background is that variability from non-physiological sources is reduced.

The noise background may be pink noise background: S(f)=A*f ̂(−B), 0<B<2. An advantage of normalizing PSDs to pink noise background is that this function generally provides the best estimate of the noise background frequency distribution.

The noise background may also be estimated by an exponential function or a polynomial.

In step f) the state of the CNS is be assessed based on the recorded spatiotemporal fluctuations, which may define a location in a predefined state space.

The recorded spatiotemporal fluctuations may be spike trains.

The recorded spatiotemporal fluctuations may be represented by PSDs.

The recorded spatiotemporal fluctuations may be represented by CSDs.

The predefined state space is defined by a set of feature vectors.

The steps may be carried out in an arbitrary order.

The subject may be an animal or a human. The animal may e.g. be a bird, a fish, such as a zebra fish, or a rodent, such as a rat.

The electrophysiological recordings may originate from recordings with different types of electrodes.

The anatomical structures may be any anatomical structure of the CNS. Examples of such anatomical structures are rostral forelimb area (RFA), primary motor cortex (M1), dorsolateral striatum (DLS), dorsomedial striatum (DMS), globus pallidus (GP), thalamus (Thal), subthalamic nucleus (STN), substantia nigra pars reticulata (SNr) and cortico-basal ganglia-thalmaic neuronal circuit.

Optionally, the method may comprise a step of low-pass filtering of the signals. This step is preferably performed between steps b) and step c). The low-pass filtering of the signals may be performed with a cut-off frequency of 100 to 1000 Hz, preferably 200 to 500 Hz, and most preferably 300 Hz.

According to one embodiment, the method may further comprise the step of e3) reducing noise and variability by averaging PSDs from different electrode pairs from the same anatomical structure. This step may be performed by calculating PSDs for the differential measures from all unique pairs of electrodes and then averaging those PSDs. An advantage of performing this step is that occasional particularities of individual electrodes are averaged out.

According to a second embodiment of the present invention, the set of feature vectors of the predefined state space may have been defined by features of signals obtained in electrophysiological recordings under at least two reference states. An advantage of performing this step is that the reference states can be used to calibrate the state space between several recordings and/or subjects by alignment of the feature vectors representing the reference states through vector transformation.

Alternatively, the set of feature vectors of the predefined state space may have been defined by features of signals obtained in electrophysiological recordings under at least three reference states. An advantage of performing this step is that the reference states can be used to calibrate the state space between several recordings and/or subjects.

According to a third embodiment, a feature vector may have been projected onto a subspace spanned by at least two of the reference states. This may be performed by multiplying the feature vector with the projection matrix defined by the reference states. An advantage of this embodiment is that feature vectors from several recordings and/or subjects can be compared.

According to a fourth embodiment, the set of feature vectors of the predefined state space may have been defined by action potentials and/or local field potentials (LFPs) obtained in electrophysiological recordings under at least three reference states, wherein the action potentials and/or local field potentials (LFPs) may have been subjected to the steps of a) amplifying recorded action potentials and/or local field potentials; b) digitizing recorded action potentials and/or local field potentials; c) reducing the influence of current dipoles located far away from the electrodes to obtain signals that are decoupled from other anatomical structures. The action potentials and/or local field potentials (LFPs) may further have been subjected to at least one of the following five steps: d1) high-pass filtering of the signals and amplitude thresholding at a predefined value above noise background for the identification of action potentials; d2) obtaining at least one spike train from trains of action potentials generated from groups of neurons or individual cells; e1) obtaining power spectral densities (PSDs) or cross-spectral densities for the LFPs; e2) normalizing PSDs to noise background; e3) reducing noise and variability by averaging PSDs from different electrode pairs from the same anatomical structure. The feature vectors may have been transformed by a coefficient matrix obtained from principal component analysis (PCA) or related methods.

Steps a)-e3) may be as above.

An advantage of using PCA is to reduce the dimensionality of the data.

The coefficient matrix may be calculated from data from one recording. The matrix may then used to transform feature vectors from the same recording. This guarantees that the space spanned by the PCs is optimized for that recording.

The coefficient matrix may be calculated from data from one subject. The matrix may then be used to transform feature vectors from the same subject. This guarantees that the space spanned by the PCs is optimized for all recordings from that subject.

The coefficient matrix may be calculated from data from one recording, but only using data belonging to selected reference states. The matrix may then be used to transform any feature vector from the same recording. This allows for comparisons between recordings even when some inter-recording variability is present.

The coefficient matrix may be calculated from data from one subject, but only using data belonging to selected reference states. The matrix may then be used to transform any feature vector from the same subject. This allows for comparisons between subjects even when some inter-subject variability is present.

The coefficient matrix may be calculated from a representative data set. The matrix may then be used to transform any feature vector from any recording or subject. This is best when variability between recordings or subjects is negligible.

Signals obtained from electrophysiological recordings may be divided into action potentials and local field potentials (LFPs). In turn, spike trains and/or peristimulus time histograms (PSTHs) may be derived from the action potentials. Evoked potentials, as well as PSDs and cross-spectrum densities (CSDs) may be derived from the LFPs. All of these may be used to interpret the recorded signals.

According to a second aspect of the present invention, a method for assessment of a state of the central nervous system (CNS) of a subject and/or differentiation between states of the central nervous system (CNS) of a subject at a specific time point based on electrophysiological recordings of signals from at least two anatomical structures is provided. The signals are recorded from recording sites located in the anatomical structures. The electrophysiological recordings comprise spatiotemporal fluctuations in the recorded extracellular potential. The electrophysiological recordings are action potentials and/or local field potentials (LFPs) in the anatomical structures and represent the state of the CNS at said specific timepoint, wherein the method comprises the steps of a′) providing a stimulus to the CNS; a) amplifying recorded action potentials and/or local field potentials; b) digitizing recorded action potentials and/or local field potentials; c) reducing the influence of current dipoles located far away from the recording site to obtain signals that are decoupled from other anatomical structures. The method further comprises at least one of the following four steps: d1) high-pass filtering of the signals and amplitude thresholding at a predefined value above noise background for the identification of action potentials; d2) obtaining at least one spike train from trains of action potentials generated from groups of neurons or individual cells; d3) creating peristimulus time histograms (PSTHs) from spike trains; g) creating evoked potentials (EPs) from the LFPs. The method further comprises the step of f) assessing the state of the CNS based on the recorded spatiotemporal fluctuations defining a location in a predefined state space, wherein the predefined state space is defined by a set of feature vectors.

Steps a)-e3) may be as above.

By providing a stimulus, voltage fluctuations may be recorded under conditions that involve specific events or perturbations influencing the recorded activity patterns. Assessing the state of the CNS in relation to specific stimuli may reveal state specific evoked responses. For example, the response of the CNS to somatosensory stimulation may differ in a pain condition.

The stimulus provided in step a′) may be any kind of stimulus, such as an auditory stimulus, a visual stimulus, an olfactory stimulus, a taste stimulus, a somatosensory stimulus, electrical stimulation of neuronal tissue or a behavioral event.

The stimulus may be a single stimulus.

The stimulus may be a series of repeated stimuli, such as a series of discrete sound pulses.

The stimulus may constitute a specific event or action relating to the behavior of the subject.

Alternatively, step a′) may be omitted. In such a case recorded voltage fluctuations may be obtained under recording conditions that do not involve specific events or perturbations influencing the recorded activity patterns. In such cases the spontaneous brain activity is recorded and analysed. The recorded voltage fluctuations may be recorded in relation to a certain behaviour, e.g. active or inactive behavioral states or specific motor acts.

Step d3) may be performed using any method for creating peristimulus time histograms (PSTHs) from spike trains. One example of such a method is to bin spike times relative to stimulus onset for all applied stimuli.

Step g) may be performed using any method for creating evoked potentials (EPs) from the LFPs. One example of such a method is to average the LFPs time-locked to the applied stimuli. Preferably, this step is performed by subtracting the pre-stimulus baseline.

In step f) the state of the CNS is be assessed based on the recorded spatiotemporal fluctuations, which may define a location in a predefined state space.

The recorded spatiotemporal fluctuations may be PSTHs.

The recorded spatiotemporal fluctuations may be EPs.

The predefined state space is defined by a set of feature vectors.

The steps may be carried out in an arbitrary order.

The subject may be an animal or a human. The animal may e.g. be a bird, a fish, such as a zebra fish, or a rodent, such as a rat.

The electrophysiological recordings may originate from recordings with different types of electrodes.

The anatomical structures may be any anatomical structure of the CNS. Examples of such anatomical structures are rostral forelimb area (RFA), primary motor cortex (M1), dorsolateral striatum (DLS), dorsomedial striatum (DMS), globus pallidus (GP), thalamus (Thal), subthalamic nucleus (STN), substantia nigra pars reticulata (SNr) and cortico-basal ganglia-thalmaic neuronal circuit.

Optionally, the method may comprise a step of low-pass filtering of the signals. This step is preferably performed between steps b) and step c). The low-pass filtering of the signals may be performed with a cut-off frequency of 100 to 1000 Hz, preferably 200 to 500 Hz, and most preferably 300 Hz.

Only PSTHs may be used for the assessment of a state of the central nervous system (CNS) and/or for differentiation between states of the central nervous system (CNS). An advantage of using PSTHs is that state dependent changes relating to the firing of specific cell groups can be included and that test conditions favorable for differentiation of states can be explored.

Only EPs may be used for the assessment of a state of the central nervous system (CNS) and/or for differentiation between states of the central nervous system (CNS). An advantage of using EPs is that state dependent changes relating to synchronized population activity can be included and that test conditions favorable for differentiation of states can be explored.

Both PSTHS and EPs may be used for the assessment of a state of the central nervous system (CNS) and/or for differentiation between states of the central nervous system (CNS). An advantage of using both PSTHs and EPs is that different aspects of the neuronal information processing can be assessed. Generally a combination of these measures will result in a more detailed assessment of the neuronal information processing than any of the measures independently.

According to one embodiment, the set of feature vectors of the predefined state space may have been defined by features of signals obtained in electrophysiological recordings under at least two reference states. An advantage of performing this step is that the reference states can be used to calibrate the state space between several recordings and/or subjects by alignment of the feature vectors representing the reference states through vector transformation.

Alternatively, the set of feature vectors of the predefined state space may have been defined by features of signals obtained in electrophysiological recordings under at least three reference states. An advantage of performing this step is that the reference states can be used to calibrate the state space between several recordings and/or subjects.

According to another embodiment, a feature vector has been projected onto a subspace spanned by at least two of the reference states. This may be performed by multiplying the feature vector with the projection matrix defined by the reference states. An advantage of this embodiment is that feature vectors from several recordings and/or subjects can be compared.

According to another embodiment, the set of feature vectors of the predefined state space may have been defined by action potentials and/or local field potentials (LFPs) obtained in electrophysiological recordings under at least three reference states. A stimulus may have been provided to the CNS prior to the electrophysiological recordings. The action potentials and/or local field potentials (LFPs) may have been subjected to the steps of a) amplifying recorded action potentials and/or local field potentials; b) digitizing recorded action potentials and/or local field potentials; c) reducing the influence of current dipoles located far away from the electrodes to obtain signals that are decoupled from other anatomical structures. The action potentials and/or local field potentials (LFPs) may further have been subjected to at least one of the following four steps: d1) high-pass filtering of the signals and amplitude thresholding at a predefined value above noise background for the identification of action potentials; d2) obtaining at least one spike train from trains of action potentials generated from groups of neurons or individual cells; d3) creating peristimulus time histograms (PSTHs) from spike trains; g) creating evoked potentials (EPs) from the LFPs. The feature vectors may have been transformed by a coefficient matrix obtained from principal component analysis (PCA) or related methods.

Steps a)-d3) and g) may be as above.

An advantage of using PCA is to reduce the dimensionality of data.

The coefficient matrix may be calculated from data from one recording. The matrix may then used to transform feature vectors from the same recording. This guarantees that the space spanned by the PCs is optimized for that recording.

The coefficient matrix may be calculated from data from one subject. The matrix may then be used to transform feature vectors from the same subject. This guarantees that the space spanned by the PCs is optimized for all recordings from that subject.

The coefficient matrix may be calculated from data from one recording, but only using data belonging to selected reference states. The matrix may then be used to transform any feature vector from the same recording. This allows for comparisons between recordings even when some inter-recording variability is present.

The coefficient matrix may be calculated from data from one subject, but only using data belonging to selected reference states. The matrix may then be used to transform any feature vector from the same subject. This allows for comparisons between subjects even when some inter-subject variability is present.

The coefficient matrix may be calculated from a representative data set. The matrix may then be used to transform any feature vector from any recording or subject. This is preferred when variability between recordings or subjects is negligible.

The following embodiments relate to methods using one or more of spike trains, PSDs, CSDs, EPs and PSTHs.

According to one embodiment, each state of the CNS may be identified as being one of at least three reference states. The classification of a CNS state can help characterizing an experimental treatment.

According to another embodiment, the action potentials and/or local field potentials may have been obtained from an awake animal or human. Several CNS processes can only be investigated in an awake subject.

According to yet another embodiment, the action potentials and/or local field potentials may have been obtained from at least one anatomical structure located below the superficial structures of the brain. Examples of such structures are rostral forelimb area (RFA), primary motor cortex (M1), dorsolateral striatum (DLS), dorsomedial striatum (DMS), globus pallidus (GP), thalamus (Thal), subthalamic nucleus (STN), substantia nigra pars reticulata (SNr) and cortico-basal ganglia-thalmaic neuronal circuit.

According to a third aspect of the present invention a method according to the present invention is used for assessment of a state of the CNS and/or differentiation of at least two states of the CNS. The use of the invention for classification of a CNS state can help characterizing an experimental treatment.

According to a fourth aspect of the present invention a method according to the present invention is used for evaluating the effect of a treatment of a condition or disease, wherein the condition or disease is neurological and/or psychiatric. A neurophysiological description is useful for the development of treatments for such conditions.

According to one embodiment, the condition may be Parkinson's disease. A neurophysiological description is useful for the development of treatments for Parkinson's disease.

According to one embodiment, the condition may be schizophrenia. A neurophysiological description is useful for the development of treatments for schizophrenia.

According to one embodiment, the condition may be a pain condition. A neurophysiological description is useful for the development of treatments for pain conditions.

According to one embodiment, the condition may be levodopa-induced dyskinesia. A neurophysiological description is useful for the development of treatments for levodopa-induced dyskinesia.

According to another aspect of the present invention, a system for assessment of a state of the central nervous system (CNS) of a subject and/or differentiation between states of the central nervous system (CNS) of a subject at a specific time point based on electrophysiological recordings of signals from at least two anatomical structures is also provided. The signals are recorded from recording sites located in the anatomical structures. The electrophysiological recordings comprise spatiotemporal fluctuations in the recorded extracellular potential. The electrophysiological recordings are action potentials and/or local field potentials (LFPs) in the anatomical structures and represent the state of the CNS at said specific timepoint. The system comprises means for amplifying recorded action potentials and/or local field potentials; means for digitizing recorded action potentials and/or local field potentials; means for reducing the influence of current dipoles located far away from the recording site to obtain signals that are decoupled from other anatomical structures. The system further comprising at least one of the following four means: means for high-pass filtering of the signals and amplitude thresholding at a predefined value above noise background for the identification of action potentials; means for obtaining at least one spike train from trains of action potentials generated from groups of neurons or individual cells; means for obtaining power spectral densities (PSDs) or cross-spectral densities for the LFPs; means for normalizing PSDs to noise background. The system further comprises means for assessing the state of the CNS based on the recorded spatiotemporal fluctuations defining a location in a predefined state space. The predefined state space is defined by a set of feature vectors.

In short, by using the information comprised in the recorded spatiotemporal fluctuations, the state of the CNS at a given time point may be localized in a state space. Depending on the location of the state in the state space, conclusions regarding the state of the CNS may be drawn.

According to one embodiment, the system may further comprise means for reducing noise and variability by averaging PSDs from different electrode pairs from the same anatomical structure. This step may be performed by calculating PSDs for the differential measures from all unique pairs of electrodes and then averaging those PSDs. An advantage of performing this step is that occasional particularities of individual electrodes are averaged out.

According to another aspect of the present invention, a system for assessment of a state of the central nervous system (CNS) of a subject and/or differentiation between states of the central nervous system (CNS) of a subject at a specific time point based on electrophysiological recordings of signals from at least two anatomical structures is also provided. The signals are recorded from recording sites located in the anatomical structures. The electrophysiological recordings comprise spatiotemporal fluctuations in the recorded extracellular potential. The electrophysiological recordings are action potentials and/or local field potentials (LFPs) in the anatomical structures and represent the state of the CNS at said specific timepoint. The system comprises means for amplifying recorded action potentials and/or local field potentials; means for digitizing recorded action potentials and/or local field potentials; means for reducing the influence of current dipoles located far away from the recording site to obtain signals that are decoupled from other anatomical structures. The system further comprises at least one of the following four means: means for high-pass filtering of the signals and amplitude thresholding at a predefined value above noise background for the identification of action potentials; means for obtaining at least one spike train from trains of action potentials generated from groups of neurons or individual cells; means for creating peristimulus time histograms (PSTHs) from spike trains; means for creating evoked potentials (EPs) from the LFPs. The system further comprises means for assessing the state of the CNS based on the recorded spatiotemporal fluctuations defining a location in a predefined state space. The predefined state space is defined by a set of feature vectors.

In one embodiment, the system further comprises means for providing a stimulus to the CNS. Such a means may be a loudspeaker, a light source, a device for providing somatosensory stimuli, such as a mechanical tap or a CO₂-laser, or a device for providing electrical stimulation of neuronal tissue.

The following embodiments relate to all systems according to the present invention.

According to one embodiment, the system may further comprise means defining the set of feature vectors of the predefined state space based on features of signals obtained in electrophysiological recordings under at least three reference states. An advantage of this feature is that the reference states can be used to calibrate the state space between several recordings and/or subjects.

According to one embodiment, the system may further comprise means for projecting a feature vector onto a subspace spanned by at least one of the reference states. This may be performed by multiplying the feature vector with the projection matrix defined by the reference states. An advantage of this embodiment is that feature vectors from several recordings and/or subjects can be compared.

According to one embodiment, the system may further comprise atleast three electrodes. This enables independent referencing when at least two anatomical structures are recorded in parallel.

The system may comprise up to 64 electrodes.

The system may comprise up to 128 electrodes.

The system may comprise up to 1024 electrodes.

The electrodes may be arranged in electrode arrays. Each electrode array may comprise 2 or more individual electrodes, preferably 5-20 individual electrodes. Multiple measurements within an anatomical structure increases signal reliability.

In each electrode array, the individual electrodes may be arranged spatially in a predefined pattern. In this way, a precise location of the individual electrodes may be obtained. This facilitates the analysis of spatial current-source density estimation.

In each electrode array, the individual electrodes may be arranged spatially in a random pattern. This arrangement may facilitate implantation procedures by allowing for bundling of electrodes in a group without a specific internal organization.

The system may comprise 1-100 electrode arrays. Preferably, the system may comprise 8-20 electrode arrays.

According to another embodiment, each electrode has a diameter up to 100 μm and each electrode is stiff enough to penetrate into the anatomical structure.

According to yet another embodiment, each electrode has a diameter up to 100 μm and at least one electrode is flexible in at least one dimension.

According to one embodiment, the system may further comprise a recording device being able to be connected to at least three electrodes and having the ability to record action potentials and/or local field potentials in said anatomical structure, wherein the recorded action potentials and/or local field potentials at one specific timepoint represent the state of the CNS at said specific timepoint.

According to another aspect of the present invention, a system according to the present invention is used for assessment of a state of the CNS and/or differentiation of at least two states of the CNS. The use of the system for classification of a CNS state can help characterizing an experimental treatment.

According to another aspect of the present invention, a system according to the present invention is used for evaluating the effect of a treatment of a condition or disease, wherein the condition or disease is neurological and/or psychiatric. The use of the system to obtain a neurophysiological description is useful for the development of treatments for such conditions.

According to one embodiment, the condition may be Parkinson's disease. The use of the system to obtain a neurophysiological description is useful for the development of treatments for Parkinson's disease.

According to one embodiment, the condition may be schizophrenia. The use of the system to obtain a neurophysiological description is useful for the development of treatments for schizophrenia.

According to one embodiment, the condition may be a pain condition. The use of the system to obtain a neurophysiological description is useful for the development of treatments for pain conditions.

According to one embodiment, the condition may be levodopa-induced dyskinesia. The use of the system to obtain a neurophysiological description is useful for the development of treatments for levodopa-induced dyskinesia.

Other objectives, features and advantages of the present invention will appear from the following detailed disclosure, from the attached claims, as well as from the drawings. It is noted that the invention relates to all possible combinations of features.

Generally, all terms used in the claims and the following description are to be interpreted according to their ordinary meaning in the technical field, unless explicitly defined otherwise herein. All references to “a/an/the [system, device, component, means, step, etc.]” are to be interpreted openly as referring to at least one instance of said system, device, component, means, step, etc., unless explicitly stated otherwise.

As used herein, the term “comprising” and variations of that term are not intended to exclude other additives, components, integers or steps. The method described herein is not limited to use in connections with the system described herein. That is to say, the method may be implemented using any suitable system or separate components.

### Definitions

With intervention, as used herein, we refer to pharmacological interventions and interventions based on electrical stimulation.

With interventions based on electrical stimulation, as used herein, we refer to electrical stimulation of neuronal tissue of the central and peripheral nervous system.

With system level state, as used herein, we refer to the physiological state of the CNS as defined by the neuronal activity patterns obtained in recordings from several (at least two) separate anatomical structures of the brain simultaneously.

With action potential, as used herein, we refer to a transient change in membrane potential of a neuron caused by rapid changes in conductace of Na/K ions through voltage dependent channels.

With local field potential, as used herein, we refer to an electrical potential in a small volume of extra cellular space caused primarily by the synaptic and dedritic currents of nearby neurons.

With neuronal activity patterns, as used herein, we refer to the temporal changes in electrical potentials recorded with invasive electrodes including both action potentials and local field potentials.

With spike trains we refer to the time points of action potential events from a single or a group of neurons.

With feature vector, as used herein, we refer to the vector interpretation of all measured quantities in a multivariate measurement

With high-dimensional space, as used herein, we refer to the vector space of measured quantities, i.e. the vector space spanned by the set of all, or a sub-group of, measurements, when each multivariate measurement is interpreted as a vector. The dimensionality of the space is at most equal to the number of measured quantities, and at least two dimensions.

With subject, as used herein, we refer to an experimental animal or a human. The animal may be a mammal, such as a primate or rodent, e.g. a rat. The animal may be a fish. The animal may be a bird.

With pink noise, as used herein, we refer to any noise with a power spectral density approximated by S(f)=A*f̂(−B); where A is a constant, f the frequency and the exponent B is a constant with a value between zero and two.

With flexible electrode, as used herein, we refer to an electrode which is not stiff enough for precise insertion into nervous tissue or easily is deflected from a desired path of insertion during insertion.

With stiff electrode, as used herein, we refer to an electrode which is stiff enough for precise insertion into nervous tissue which is not easily deflected from a desired path of insertion during insertion.

With transformation, as used herein, we refer to the multiplication of a vector with a transformation matrix according to standard definitions in vector algebra.

With anatomical structure, as used herein, we refer to an anatomically and/or functionally defined part of the CNS located in a specific macroanatomical volume.

### BRIEF DESCRIPTION OF THE DRAWINGS

The above, as well as additional objects, features and advantages of the present invention, will be better understood through the following illustrative and non-limiting detailed description of embodiments of the present invention, with reference to the appended drawings, and wherein:

FIG. 1 shows spatiotemporal voltage fluctuations in the recordings from different electrodes [V(t)] which are subdivided in the further signal analyses to generate separate sets of feature vectors. For the detection of action potentials, the signal is high-pass filtered and thresholded. For the further analyses of this unit activity, time-series of action potentials constituting spike trains are analysed during different conditions and/or in relation to specific events/stimuli by analyses of peri-stimulus time histograms (PSTHs) of spike times. For analyses of the local field potentials (LFPs) the signal is examined in the frequency and/or time domain. In the frequency domain—following Fourier transformation (FT) of the recorded time series—the power spectral density (PSD) in each recording electrode and/or cross-spectrum densities (CSDs) between recording channels are calculated. For the characterization of evoked activity in relation to specific events/stimuli time domain analyses are preferred, typically by analysis of the average evoked potential (EP) of a number of similar events occuring over time. Following the creation of all or a subset of these measures the resulting feature vectors are combined into a compound feature vector describing a state.

FIG. 2 shows example power spectra for local field potentials recorded under three different conditions (healthy, parkinsonian and dyskinetic) under at least 30 min, shown for three example structures (Primary Motor cortex, Dorsolateral Striatum and Subthalamic Nucleus). The relative power of different frequencies is shown following normalization of the spectra to a pink noise distribution. It can be noted that the power spectra obtained in the three different conditions differ somewhat in all three structures.

FIG. 3 shows an example state description in two dimensions for a rat recorded in three different conditions (healthy, parkinsonian and dyskinetic). The neurophysiological state of the animal was measured during 8 s recording periods throughout the experiment where feature vectors were constructed from power spectral densities of local field potentials recorded in eight different parts of the cortico-basal ganglia-thalamic loop. The x-axis represents the spectral mean difference vector between the healthy and the parkinsonian state and the y-axis represents the orthogonal part of the difference vector between the parkinsonian and dyskinetic state. It can be noted that the three states are easily identified as three separate clusters in this representation.

FIG. 4 shows an example state description in two dimensions for a rat recorded in five different conditions (healthy, parkinsonian, dyskinetic, [dyskinetic+8-OH-DPAT] and [dyskinetic+8-OH-DPAT+WAY100635]). The neurophysiological state of the animal was measured during 8 s recording periods throughout the experiment where feature vectors were constructed from power spectral densities of local field potentials recorded in eight different parts of the cortico-basal ganglia-thalamic loop. The x-axis represents the spectral mean difference vector between the healthy and the parkinsonian state and the y-axis represents the orthogonal part of the difference vector between the parkinsonian and dyskinetic state. It can be noted that the five states are easily identified as five separate clusters in this representation and that the 5-HT antagonist WAY100635 reversed the effect of 8-OH-DPAT inducing a state that closely resembles the initial dyskinetic state.

FIG. 5 shows an example state description in two dimensions for a rat recorded in four different conditions (parkinsonian, dyskinetic, [dyskinetic+8-OH-DPAT] and [dyskinetic+8-OH-DPAT+WAY100635]). The neurophysiological state of the animal was measured during 8 s recording periods throughout the experiment where feature vectors were constructed from the relative firing rates of neurons recorded in eight different parts of the cortico-basal ganglia-thalamic loop in the lesioned hemisphere. The x-axis represents the mean difference vector between the dyskinetic and the parkinsonian state and the y-axis represents the orthogonal part of the difference vector between the parkinsonian and the 8-OH-DPAT treated state. It can be noted that the four states are easily identified as four separate clusters in this representation and that the 5-HT antagonist WAY100635 reversed the effect of 8-OH-DPAT to a state closely resembling the initial dyskinetic state (represented by the two clusters to the right).

FIG. 6 shows example power spectra for local field potentials recorded under three different conditions (untreated, LSD treated and Ketamine treated) shown for three example structures (Hippocampus, Nucleus Accumbens and Thalamus). The relative power of different frequencies is shown following normalization of the spectra to a pink noise distribution. It can be noted that the power spectra obtained in the three different conditions show certain dissimilarities in all three structures but that the two psychosis-like states both are characterized by a high-frequency narrow band oscillation in n. Accumbens.

FIG. 7 shows example power spectra for local field potentials recorded under three different conditions (untreated, MK-801 treated and [MK-801+Haloperidol] treated) shown for three example structures (Hippocampus, Nucleus Accumbens and Thalamus). The relative power of different frequencies is shown following normalization of the spectra to a pink noise distribution. It can be noted that the power spectra obtained in the three different conditions show certain dissimilarities in all three structures but also that Haloperidol partly normalizes the MK-801 treated state (for example 40-100 Hz in Thalamus).

FIG. 8 shows an example state description in two dimensions for a rat recorded in five different conditions (treatment with Ketamine, LSD, MK-801, [MK-801+Haloperidol] and untreated Baseline). The neurophysiological state of the animal was measured during 8 s recording periods throughout the experiments where feature vectors were constructed from power spectral densities of local field potentials recorded in six different diencephalic/telencephalic regions. The x-axis represents the spectral mean difference vector between the untreated and the Ketamine treated state and the y-axis represents the orthogonal part of the difference vector between the untreated and the LSD treated state. It can be noted that the five states can be identified as five clusters in this representation and that the antipsychotic drug Haloperidol largely reversed the state induced by MK-801 back towards the untreated state.

### DESCRIPTION OF EMBODIMENTS OF THE INVENTION

Described herein is a method for characterization and differentiation between states of the central nervous system, based on multi-structure electrophysiological recordings. In particular the embodiments relate to a method to characterize CNS states induced by disease or models of disease and/or experimental interventions aimed at modifying these states and/or treating disease.

One embodiment comprises the characterization and/or differentiation between states of the central nervous system in a subject, wherein the method encompasses at least one multi-structure electrophysiological recording.

One embodiment comprises a platform for evaluation of different therapeutic approaches used in the treatment of neurologic and psychiatric disease and information on how and why interventions may have beneficial effects, based on their influence on activity patterns in interconnected CNS sub-systems.

One embodiment relates to an analytical method that in a high-dimensional space describes the most relevant CNS systems level state changes created through interventions in order to help driving the nervous system towards a healthy state. An aspect of this embodiment includes multivariate analyses of the electrophysiological recording, such as recorded voltage fluctuations, that allow the investigator to extract the most informative part of the brain activity patterns under specific experimental conditions and to provide a meaningful interpretation of large data sets. Another aspect comprises adaptation of the analytical methods and experimental procedures to each particular research question in order to effectively explore the space of possible CNS activity states relevant for that disease/intervention. It is also an aspect that analytical methods are designed to work equally well without any prior knowledge of what electrophysiological changes that might occur, enabling efficient analyses of novel drug candidates and other experimental interventions.

A further aspect is that CNS conditions that may not induce overt signs in experimental in vivo animal models of disease (such as psychosis, mood disorders, autism spectrum disorders, pain states, etc.) can be investigated based on CNS systems level state characterizations following, for example, drug manipulation known to induce similar states in humans.

Recording Interface:

An aspect is a recording interface that allows for extracellular recordings of action potentials and LFPs over several weeks. Although optical techniques based on, for example, voltage sensitive dyes or indirect measures of dynamics in intracellular Ca²⁺-concentrations are feasible methodological approaches to sampling neuronal activity, a preferred embodiment of the invention comprises the use of physical recording electrodes placed in the neuronal tissue.

For this purpose, the tip of the electrode has to be relatively small (<100 μm in diameter). In one embodiment, stiff electrodes are used, the electrode must be stiff enough to penetrate through the pia mater and underlying brain tissue to be inserted into small CNS targets located deep down in the brain. According to one embodiment, the electrode, which is thin (10-50 μm) is comprised of at least two (in some embodiments >100) polymer-insulated metal wire electrodes that are used as the electrode interface to the nervous tissue. Such wires are commercially available. Conductive metals like stainless steel, noble metals and alloys are possible materials and in some embodiments the material is tungsten, due to its inherent stiffness, allowing for implantation of wires only a few tenths of microns thick, e.g. a wire diameter of ˜30 μm.

In another embodimentflexible electrodes are used, the electrode is not stiff enough for precise insertion into nervous tissue or easily is deflected from a desired path of insertion during insertion.

Three-Dimensional Arrangement of Recording Wires:

To obtain sufficient precision in the positioning and relative arrangement of the different electrode wires, methods designed to control the alignment of the electrode wires is required. Preferred methods for this procedure are described below.

It is an aspect that the recording electrode, comprising the arranged recording wires, is chronically implanted to allow for recording during long periods in freely behaving subjects.

Which brain structures that are most desirable to record from differ depending on the disease condition or treatment intervention that is being investigated. In the example applications further described below (see Experimental section) pharmacological interventions aimed at treating both neurologic (Parkinson's disease and/or levodopa-induced dyskinesia) and psychiatric conditions (psychosis) are analyzed. In these embodiments several structures in the cortico-basal ganglia-thalamic loop have been targeted in order to analyze Parkinson's disease and levodopa-induced dyskinesia and several cognitive/limbic structures together with thalamic nuclei have been used for assessment of the psychotic state.

According to other embodiments, other targets could be used in order to analyze other disease conditions, and it is considered to be within the capacity of those skilled in the art to arrange the implant appropriately.

Exploring Different States:

To obtain a comprehensive description of systems level electrophysiological states under specific conditions, reference states need to be defined. These states/conditions should be chosen to fit the specific disease/intervention that is being investigated.

In one example application, described below, anti-dyskinetic interventions in levodopa-induced dyskinesia are shown, this embodiment includes: 1) a healthy state, 2) a Parkinsonian state (created via neurotoxic injections of 6-hydroxydopamine [6-OHDA] to the medial forebrain bundle in a rat—a procedure known to have neurotoxic effects on midbrain dopaminergic neurons), 3) a dyskinetic state (created by levodopa-treatment of the Parkinsonian rat). These states are compared to the states induced by a number of different pharmacological interventions in the dyskinetic rat. Those skilled in the art would know how to modify this in view of other disease conditions.

Analytical Approach to Definition and Assessment of CNS States:

The brain signals recorded in each recording wire under different conditions are used to construct a high-dimensional representation of the electrophysiological features characterizing the different states. These features include frequency and timing of action potentials and LFP-fluctuations in the different recording channels as well as higher order interactions between channels such as correlations in time. Importantly, signals are not analyzed only in the time domain (spike trains, PSTHs, EPs) but also in the frequency domain (PSDs, CSDs) allowing for characterizations based on relative changes in spectral power or coherence and phase between signals recorded in different electrodes. To extract spectral features deviating from the background distribution a normalization to an exponential or power distribution is beneficial. In this context normalization to a pink noise distribution is preferred (that is a 1/f like distribution).

Calculating PSDs for the differential measures from all unique pairs of electrodes and then averaging those PSDs is preferred.

Computational methods, such as principal component analysis, are then applied to extract the part of the signals that can be used to most effectively differentiate between states. Other related methods such as non-negative matrix factorization or factor analysis are also feasible.

Electrophysiological states sampled at different time periods which display relatively similar multi-structure activity patterns are used to define a CNS state. Other CNS states can then be assessed in relation to these states or other predefined CNS states. The degree of similarity between states can be quantified by their distance in the multi-dimensional space spanned by the feature vectors defining the state.

The relative power of LFP oscillations of different frequencies may also be used to characterize the different states. There are also other measures known or possible to elucidate to those skilled in the art allowing the investigator to make use of the method for other conditions.

### DETAILED DESCRIPTION OF PREFERRED EMBODIMENTS OF THE INVENTION

Construction of Recording Interface:

Commercially available, polymer-insulated 33 μm tungsten wires were used as recording and reference electrodes.

Precision positioning of the wires, targeting the desired brain structures, was made possible by design of a custom perforated plastic sheet adapted to the horizontal anatomical position of all the CNS structures to be recorded. A matrix of holes (˜100 μm in diameter) with 250 μm separation (center-to-center) was made by laser milling (coordinates for the different targets are readily available in published literature).

High-accuracy vertical positioning of each electrode tip was accomplished with the help of an appropriately designed 3D (three dimensional) plastic mold determining the vertical relative length of sub-groups of wires targeting the different structures. The plastic sheet was fixated to the top of the 3D-aligner, so that the holes of the 2D (two dimensional) array were overlapping the respective vertical spaces in the 3D-aligners for the different structures.

Tungsten has inherent properties that introduce difficulties soldering it to another metal to make electrical connection between wires and connector pins. The stiffness of the wires also tends to introduce tension when manipulating them making detailed wire positioning more challenging. To overcome this problem, a printed circuit board was designed linking wires and connector. Electrical contact to tungsten wires was by application of conductive epoxy glue, such as silver based epoxy glue, or silver paint covering the junctions between the printed circuit board and the wires.

Finally, all parts of the arranged electrode were fixed into one piece by UV-light hardening glue.

Signal Acquisition:

Local field potentials and unit activity (spikes; that is action potentials from individual neurons) can be amplified and digitized with standard equipment, for example the multi-channel system from Neuralynx Inc.

To separate the local field potentials from the wideband signal, the signal is optionally low-pass filtered with a cut-off frequency of 300 Hz. From the raw LFP time series local bipolar LFP time series are computed offline from all unique pairs of electrodes from the same structure. This will reduce the influence of current dipoles that are located far away from the electrodes, and as a consequence signals that are decoupled from other anatomical structures are obtained.

To obtain the spikes, the signal is high-pass filtered with a cut-off frequency of 600 Hz and 1-ms epochs around samples greater than 2.5 times the noise amplitude are saved for off-line sorting using standard techniques.

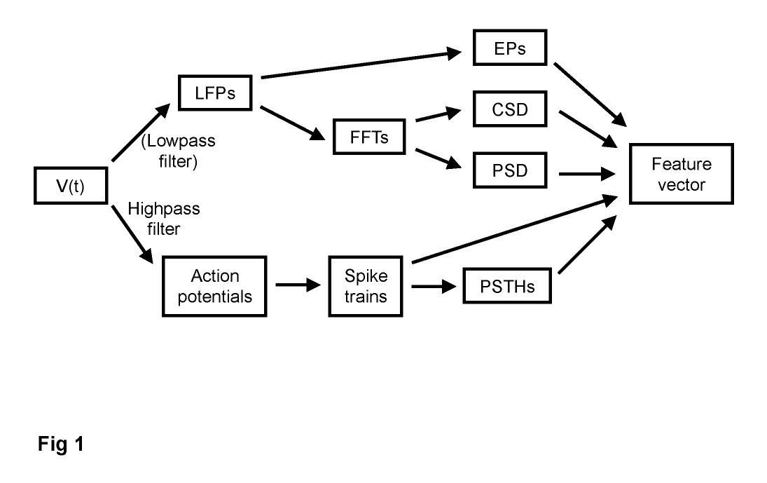

The flow chart in FIG. 1 illustrates the described embodiment of the initial signal analysis.

Frequency Analysis of LFPs:

Power spectral densities (PSDs) are calculated with a multitaper method (Pesaran, 2008) (50%-overlapping 8-s windows, 7 tapers) implemented in Chronux 2.0 (Mitra and Bokil, 2007). To stabilize the variance and to emphasize narrowband oscillations over wideband signals, PSDs are normalized to the pink noise background according to Halje et al. (Halje et al., 2012), but with one important adaptation: Due to the complexity of the data it is not practical to manually pick frequency bands with pure pink noise in all structures and conditions to get unbiased estimates of the noise background. Instead the whole frequency axis is divided into 20 logarithmically spaced bands (1-1.3, 1.3-1.7, . . . , 151.3-200 Hz) and the median power of each band is used for the fitting of the pink noise power curve S(f)=A/f^{B}. The pink noise normalization allows us to describe deviations from the pink noise floor in terms of the unit dBₚᵢₙₖ. As a final step, an average spectrogram is calculated for each structure, based on the spectrograms for each individual local bipolar LFP time series.

If frequency bands are manually selected it is important to only use frequency bands where the pink noise dominates, i.e. to avoid known electrophysiological rhythms and external oscillatory artifacts such as power line noise. The following bands can for example be used in the example application relating to levodopa-induced dyskinesia: 2-6 Hz, 15-20 Hz, 35-45 Hz, 60-70 Hz, and 105-145 Hz. To reduce noise and variability, PSDs from different electrode pairs are averaged if they come from the same anatomical structure.

Analysis of Spike Trains:

Offline spike sorting can be performed with commercially available software to obtain spike trains of individual neurons, for example Offline Sorter by Plexon Inc. Standard spike train statistics like average firing rate and coefficient of variation is then calculated and used as features in the state-space analysis.

Construction of a Systems-Level State Space:

The collection of all measures described above can be used as a description of the (steady-)state of the neural system at a given time point. Such a description can be defined as a feature vector x with

n_{structures}·n_{frequencies}+nₙₑᵤᵣₒₙₛ·nₛₚᵢₖₑₛₜₐₜₛ

elements, where each element is a measured variable. Typically, there are a couple of thousand variables in such a state description and it is necessary to reduce the dimensionality before it is practical to make further state characterizations. Principal component analysis (PCA) is used to achieve this. Thus, PCA may be used as a tool to detect the relevant treatment effects in such a high-dimensional data set. To compensate for the heterogeneity of the feature variables, the variables are weighted with the inverse of their variance. Depending on the application three different strategies are used to calculate the PC-space:
1. The principal components (PCs) are defined once per individual with the data from all available treatments/conditions. This guarantees that the space spanned by the PCs is optimized for that data set.
2. The PCs are defined once per individual, but only on a few selected reference states. This allows for comparisons between individuals even when some inter-individual variability is present.
3. The PCs are defined once on a representative data set and then used for all individuals. This is best when inter-individual variability is negligible.

Given that the original features contain sufficient information to separate between treatment states, each state will be a distinct cluster in the space spanned by the PCs. The smallest subspace of this space that still separate all clusters was used as a “state space” (cluster separation/validity is quantified using standard techniques like Dunn/Davies Bouldin Index or other suitable methods).

Comparisons Between States:

Once the state space is defined it is possible to quantify similarities and differences between states in a general framework. Let the row vector x represent any sample data point in state space, while x_{I} and x_{II} each represent a data point belonging to state I and state II, respectively. Let (x_{I}) denote the average of all samples belonging to state I. The mean difference between the states,

b= [symbol] x_{II} [symbol] − [symbol] x_{I} [symbol] ,

can be interpreted as a direction in state space, and by taking the scalar product x·b we get the projection of any sample x onto this direction. Hence, we could construct a benchmark index

$${B}_{II - I} ( x ) = \frac{( x - 〈 {x}_{I} 〉 ) \cdot b}{{‖ b ‖}^{2}}$$

with the intuitive geometrical interpretation of being the projection onto the mean state difference, while fulfilling B_{II-I}( [symbol] x_{I} [symbol] )=0 and B_{II-I}( [symbol] x_{II} [symbol] )=1.

Comparisons Between Recordings or Subjects:

In electrophysiological recordings some sources of variance are difficult to control for experimentally. These relate to variability in brain circuit anatomy between subjects, the exact locations of the recording electrodes, signal-to-noise levels etc. Uncontrolled variance will ultimately reduce the statistical power and generalizability of an experiment. However, due to the apparent robustness of certain brain states it is possible to use these states as reference points for calibration across recordings or subjects. In mathematical terms, this calibration can be performed as a projection onto the subspace spanned by the vectors representing the reference states. Let the row vector [symbol] xᵢ [symbol] denote reference state i in one recording and the row vector [symbol] yᵢ [symbol] denote reference state i in another recording. Define the matrices P′ and P′ as the concatenation of all reference vectors of the respective recording:

$${P}^{′} = [ \begin{matrix}〈 {x}_{1} 〉 \\ 〈 {x}_{2} 〉 \\ … \\ 〈 {x}_{n} 〉\end{matrix} ] and {P}^{″} = [ \begin{matrix}〈 {y}_{1} 〉 \\ 〈 {y}_{2} 〉 \\ … \\ 〈 {y}_{n} 〉\end{matrix} ]$$

Then the projection of any vector x from the first recording and any vector y from the second recording onto the n-dimensional subspace spanned by the n reference states is given by P′x and P″y, respectively. The projected vectors P′x and P″y can then be compared directly, for example as described in the previous section.

To test the usefulness of this approach, a classifier was trained on a recording with three states (parkinsonian, dyskinetic and dyskinesia treated with amantadine). The classifier (a Gaussian mixture model) was trained in the subspace spanned by the parkinsonian and dyskinetic state and tested on a recording from a different animal. This animal had been treated with the drug amantadine which has been proposed to have certain anti-dyskinetic effects. The amantadine treated state was correctly identified 85% of the time (i.e. the true positive rate) in the new animal, compared to 89% of the time in the animal in which the classifier was trained. The corresponding false positive rates were 4% and 2%, respectively. As a comparison, the true positive rate without calibration was 8%.

### Experiments

In the following two experiments, the use of the method and system for systems level state characterization in experimental treatment in a neurologic and psychiatric condition are exemplified. These results are mere examples and should by no means be interpreted to limit the scope of the invention. Rather, the invention is limited only by the accompanying claims.

CNS States Associated with Parkinsonism and Levodopa-Induced Dyskinesia:

To characterize the CNS effects of experimental treatment of symptoms in levodopa-induced dyskinesia, the most widely used model of this disease condition was employed—the medial forebrain bundle 6-OHDA hemilesioned rat. In this model, parkinsonism is induced by the injection of this neurotoxin resulting in extensive neurotoxic lesions of midbrain dopaminergic neurons projecting to the forebrain. By injecting the drug in only one of the hemispheres the other hemisphere can be used as an internal control in different recordings conditions/behavioral situations. Following a period of levodopa treatment, animals developed dyskinetic symptoms affecting the side of the body contralateral to the brain lesion (Halje et al., 2012). Thus, neuronal recordings obtained in the parkinsonian and dyskinetic condition can be compared to a healthy control condition. Rats were chronically implanted with 128 recording electrodes according to the methods described above and CNS activity patterns were recorded in a number of experiments performed in four adult female Sprague-Dawley rats.

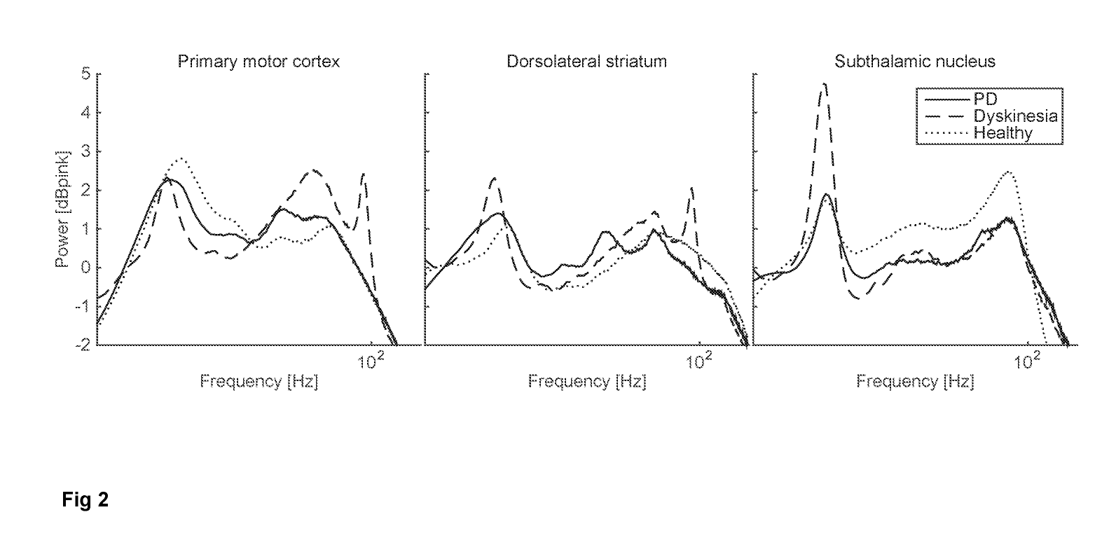

State specific neurophysiological activity patterns were observed in all recorded structures of the cortico-basal ganglia-thalamic loop during the experiments. In FIG. 2, spectra are shown illustrating the relative differences in LFP PSDs in the three states for three example structures.

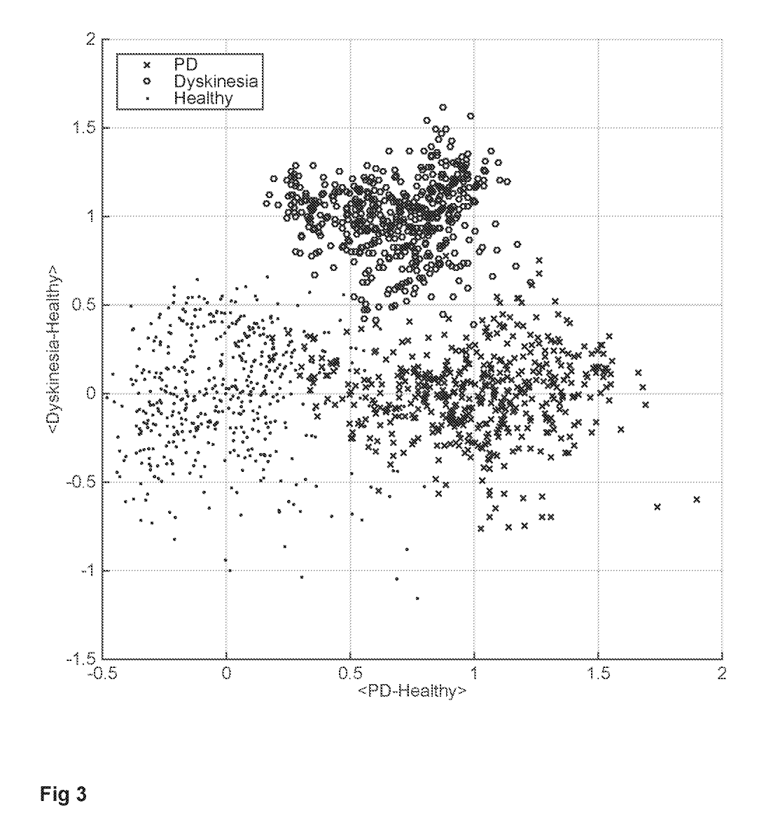

Based on the LFP PSDs, a state space was constructed according to the methods described above and 8 s-periods sampled during the different recording conditions were plotted in 2D-plots where the x-axis represents the spectral mean difference vector between the healthy and the parkinsonian state and the y-axis represents the orthogonal part of the difference vector between the parkinsonian and dyskinetic state (FIG. 3). In this representation it is evident that the three different states can be clearly differentiated.

In order to investigate the treatment effects of the 5-hydroxytryptamine receptor subtype 1A [5-HT_{1A}] agonist (8-OH-DPAT) which has previously been shown to have certain anti-dyskinetic effects in animal models of levodopa-induced dyskinesia, we administered 1 mg/kg and 0.4 mg/kg i.p. respectively, at the time point of peak dyskinesia in two experiments. The 5-HT_{1A} agonist proved to effectively suppress dyskinetic symptoms, an effect that could be fully reversed through treatment with the 5-HT_{1A}-antagonist WAY1000635.

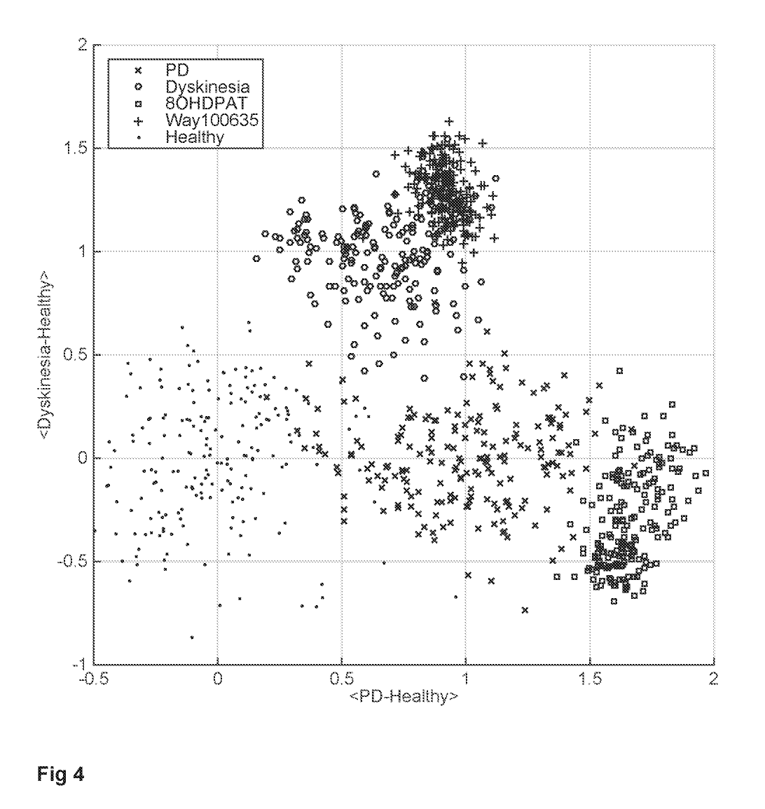

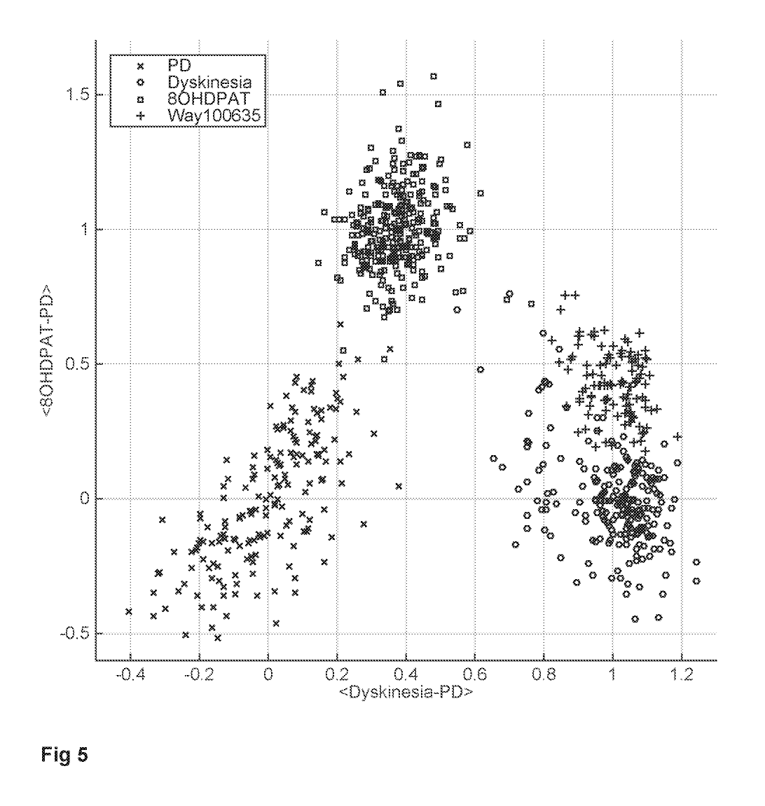

Both changes in firing rate of individual neurons and in LFP PSDs were observed in the lesioned hemisphere during the different phases of the experiment. Based on spike trains and the LFP PSDs, respectively, two state spaces were constructed according to the methods described above and 8 s-periods sampled during the different recording conditions were plotted in two-dimensional plots (using the same representation as in FIG. 3). It was noted that while the drug reduced dyskinetic symptoms and induced a CNS state that differed to the parkinsonian/dyskinetic state this state nevertheless differed to the healthy condition (FIGS. 4 and 5).

In fact, in this representation the drug primarily shifted the dyskinetic state back towards the parkinsonian state. This finding is in agreement with previous reports in behavioral experiments suggesting that the anti-dyskinetic effect of 8-OH-DPAT is associated with motor disabilities. Thus, whereas the this drug may not be a preferred choice in the treatment of dyskinesia, other drug candidates and/or combinations of drugs could potentially be evaluated with respect to their ability to restore a healthy state through the use of similar CNS state characterizations.

Note however, that each recording configuration will produce a unique state space, calling for reference manipulations that can be used to create a remapping of states if such recordings need to be pooled together in later analyses.

CNS States Associated with Psychosis:

Psychiatric diseases such as for example schizophrenia are by many researchers regarded as particularly hard to investigate in experimental animals since only a limited number of consistent changes in behavior are known to be strongly linked to specific symptoms in animal models of psychiatric conditions. Providing a neurophysiological state characterization for these types of disease conditions could therefore open up new possibilities to study the experimental treatment of these diseases.

To characterize the CNS effects of experimental treatment of symptoms in psychosis we here performed multi-structure recording in rats that were treated with pharmacological substances known ventral to induce psychosis-like states in humans. For this purpose, a number of diencephalic and telencephalic structures were recorded in parallel including: hippocampus [corresponding to anterior hippocampus in humans], nucleus accumbens [core and shell], medial prefrontal cortex and anterior gyrus cinguli (adapted to rodent anatomy), the mediodorsal nucleus and the medial geniculate nucleus in thalamus.

Two different types of pharmacological interventions known to induce psychosis-like states were investigated: 1) Antagonism of glutamatergic NMDA-receptors through systemic treatment with the dissociative anesthetics MK-801/Ketamine and 2) Activation of 5-HT_{2A}-receptors and a few other receptor groups using the classic hallucinogenic compound lysergic acid diethylamide (LSD). In some experiments the antipsychotic drug Haloperidol was subsequently applied to assess the CNS state changes induced by this experimental treatment.

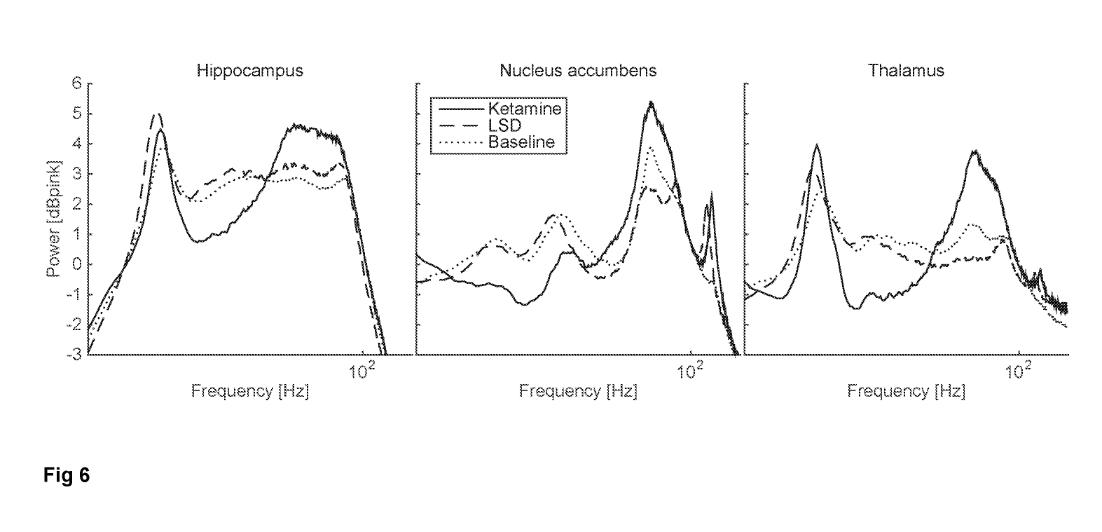

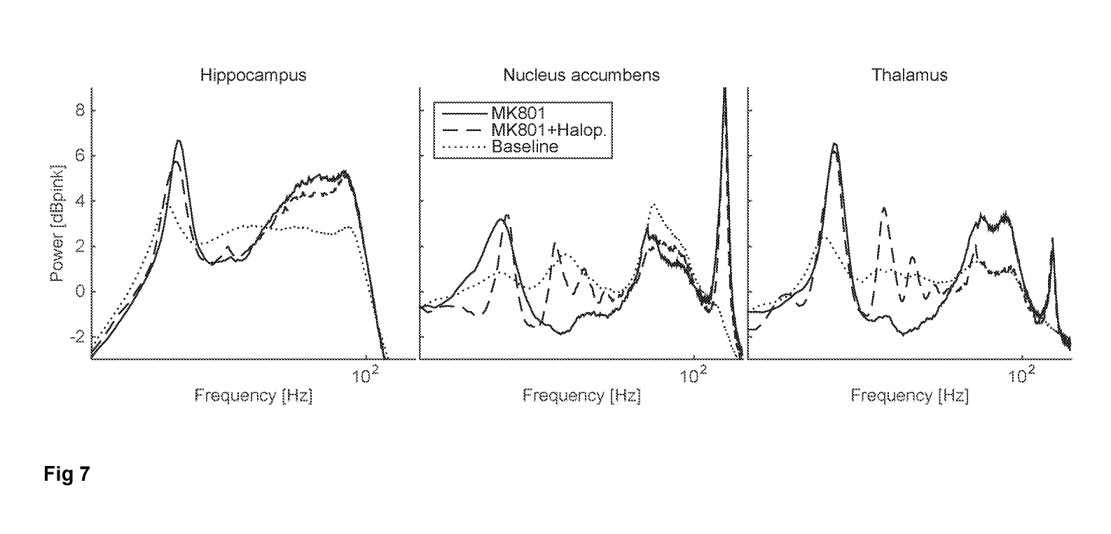

State specific neurophysiological activity patterns were observed in all recorded structures during the experiments. In FIG. 6, LFP spectra are shown for these two different animal models of psychosis illustrating the relative differences in LFP PSDs for three different states (Ketamine treated state, LSD treated state and Baseline—representing healthy control conditions) for three example structures (Hippocampus, Nucleus Accumbens and Thalamus). Similarly, in experiments investigating the CNS effects of MK-801, LFP spectra showed that the MK-801 treated state could be differentiated from Baseline conditions and that subsequent treatment with the antipsychotic drug Haloperidol altered the MK-801 treated state but could not fully restore a healthy state (FIG. 7).

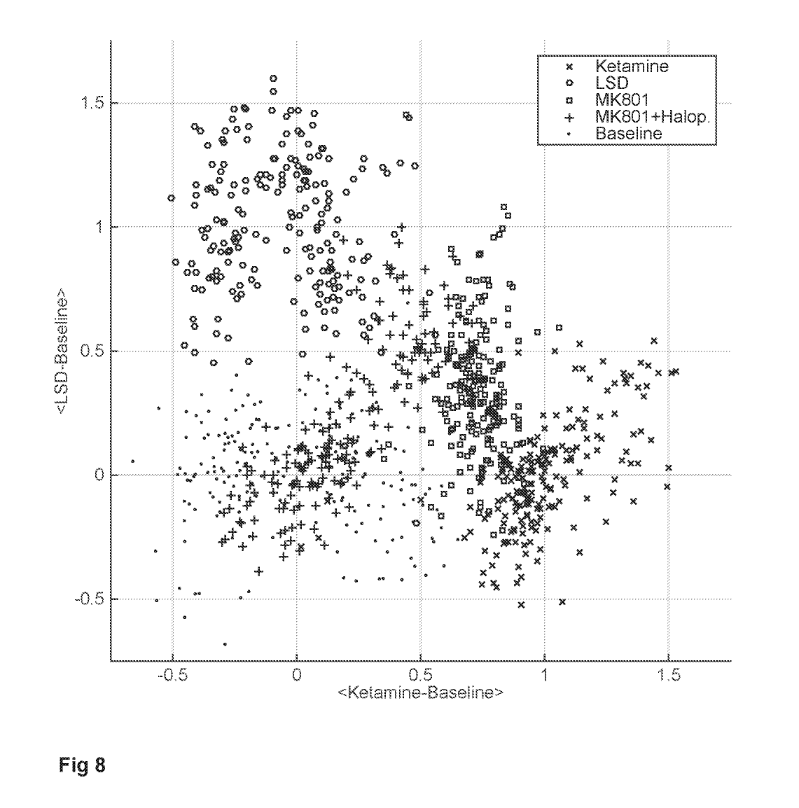

Based on the LFP PSDs, a state space was constructed according to the methods described above and 8 s-periods of neuronal activity sampled during these five different recording conditions (Ketamine, LSD, MK-801, [MK-801+Haloperidol] and Baseline) were plotted in a 2D-plot where the x-axis represents the spectral mean difference vector between the healthy and the Ketamine treated state and the y-axis represents the orthogonal part (to the x-axis) of the difference vector between the LSD treated state and the healthy state (FIG. 8). In this representation it is evident that the different states can be differentiated. Notably, the two NMDA-antagonists cluster next to each other to the right in the plane and Haloperidol shifts the MK-801 treated state close to control conditions suggesting a certain treatment effect even though control conditions were not fully restored.

In these experiments it was also noted that the behavioral changes induced by some of the drugs (for example, increased locomotion 10-50 min after injection for Ketamine) did not noticeably influence the neurophysiological state of the recorded structures, whereas the drugs per se produced obvious long-lasting changes (>1 h). This discrepancy shows that neurophysiological state characterizations can provide new information about the effects of the drugs that is not accessible in experiments that only rely on behavioral analyses.

### REFERENCES

Fanselow E E, Reid A P, Nicolelis M A (2000) Reduction of pentylenetetrazole-induced seizure activity in awake rats by seizure-triggered trigeminal nerve stimulation. J Neurosci 20:8160-8168
Fuentes R, Petersson P, Siesser W B, Caron M G, Nicolelis M A L (2009) Spinal cord stimulation restores locomotion in animal models of Parkinson's disease. Science 323:1578-1582
Gervasoni D, Lin S-C, Ribeiro S, Soares E S, Pantoja J, Nicolelis M A L (2004) Global forebrain dynamics predict rat behavioral states and their transitions. J Neurosci 24:11137-11147
Halje P, Tamte M, Richter U, Mohammed M, Cenci M a., Petersson P (2012) Levodopa-Induced Dyskinesia Is Strongly Associated with Resonant Cortical Oscillations. J Neurosci 32:16541-16551.
Mitra P, Bokil H (2007) Observed Brain Dynamics, 1st ed. Oxford University Press, USA.
Pesaran B (2008) Neural Signal Processing: Quantitative Analysis of Neural Activity Mitra P P, ed. Soc Neurosci:1-12.
Maxwell B. Santana, Par Halje, Hougelle Simplício, Ulrike Richter, Marco Aurelio M. Freire, Per Petersson, Romulo Fuentes, Miguel A. L. Nicolelis (2014) Spinal Cord Stimulation Alleviates Motor Deficits in a Primate Model of Parkinson Disease, Neuron, (84) 4: 716-722

1. A method for assessment of a state of the central nervous system (CNS) of a subject and/or differentiation between states of the central nervous system (CNS) of a subject at a specific time point based on electrophysiological recordings of signals from at least two anatomical structures, wherein the signals are recorded from recording sites located in the anatomical structures, wherein the electrophysiological recordings comprise spatiotemporal fluctuations in the recorded extracellular potential, wherein the electrophysiological recordings are action potentials and/or local field potentials (LFPs) in the anatomical structures and represent the state of the CNS at said specific timepoint, wherein the method comprises the steps of:
a) amplifying recorded action potentials and/or local field potentials;
b) digitizing recorded action potentials and/or local field potentials;
c) reducing the influence of current dipoles located far away from the recording site to obtain signals that are decoupled from other anatomical structures;
said method further comprising at least one of the following four steps
d1) high-pass filtering of the signals and amplitude thresholding at a predefined value above noise background for the identification of action potentials;
d2) obtaining at least one spike train from trains of action potentials generated from groups of neurons or individual cells;
e1) obtaining power spectral densities (PSDs) or cross-spectral densities for the LFPs;
e2) normalizing PSDs to noise background;
and said method further comprising the step of
f) assessing the state of the CNS based on the recorded spatiotemporal fluctuations defining a location in a predefined state space;
wherein the predefined state space is defined by a set of feature vectors.

2. The method according to claim 1, further comprising the step of:
e3) reducing noise and variability by averaging PSDs from different electrode pairs from the same anatomical structure.

3. A method according to claim 1, wherein the set of feature vectors of the predefined state space has been defined by features of signals obtained in electrophysiological recordings under at least two reference states.

4. A method according to claim 3, wherein a feature vector has been projected onto a subspace spanned by at least two of the reference states.

5. The method according to claim 1, wherein the set of feature vectors of the predefined state space has been defined by action potentials and/or local field potentials (LFPs) obtained in electrophysiological recordings under at least three reference states, wherein the action potentials and/or local field potentials (LFPs) have been subjected to the steps of:
a) amplifying recorded action potentials and/or local field potentials;
b) digitizing recorded action potentials and/or local field potentials;
c) reducing the influence of current dipoles located far away from the electrodes to obtain signals that are decoupled from other anatomical structures;
wherein said action potentials and/or local field potentials (LFPs) further have been subjected to at least one of the following five steps
d1) high-pass filtering of the signals and amplitude thresholding at a predefined value above noise background for the identification of action potentials;
d2) obtaining at least one spike train from trains of action potentials generated from groups of neurons or individual cells;
e1) obtaining power spectral densities (PSDs) or cross-spectral densities for the LFPs;
e2) normalizing PSDs to noise background;
e3) reducing noise and variability by averaging PSDs from different electrode pairs from the same anatomical structure.
and wherein the feature vectors have been transformed by a coefficient matrix obtained from principal component analysis (PCA) or related methods.

6. A method for assessment of a state of the central nervous system (CNS) of a subject and/or differentiation between states of the central nervous system (CNS) of a subject at a specific time point based on electrophysiological recordings of signals from at least two anatomical structures, wherein the signals are recorded from recording sites located in the anatomical structures, wherein the electrophysiological recordings comprise spatiotemporal fluctuations in the recorded extracellular potential, wherein the electrophysiological recordings are action potentials and/or local field potentials (LFPs) in the anatomical structures and represent the state of the CNS at said specific timepoint, wherein the method comprises the steps of:
a′) providing a stimulus to the CNS;
a) amplifying recorded action potentials and/or local field potentials;
b) digitizing recorded action potentials and/or local field potentials;
c) reducing the influence of current dipoles located far away from the recording site to obtain signals that are decoupled from other anatomical structures;
said method further comprising at least one of the following four steps
d1) high-pass filtering of the signals and amplitude thresholding at a predefined value above noise background for the identification of action potentials;
d2) obtaining at least one spike train from trains of action potentials generated from groups of neurons or individual cells;
d3) creating peristimulus time histograms (PSTHs) from spike trains
g) creating evoked potentials (EPs) from the LFPs;
and said method further comprising the step of
f) assessing the state of the CNS based on the recorded spatiotemporal fluctuations defining a location in a predefined state space;
wherein the predefined state space is defined by a set of feature vectors.

7. A method according to claim 6, wherein the set of feature vectors of the predefined state space has been defined by features of signals obtained in electrophysiological recordings under at least two reference states.

8. A method according to claim 7, wherein a feature vector has been projected onto a subspace spanned by at least two of the reference states.

9. The method according to claim 6, wherein the set of feature vectors of the predefined state space has been defined by action potentials and/or local field potentials (LFPs) obtained in electrophysiological recordings under at least three reference states, wherein a stimulus has been provided to the CNS prior to the electrophysiological recordings, wherein the action potentials and/or local field potentials (LFPs) have been subjected to the steps of:
a) amplifying recorded action potentials and/or local field potentials;
b) digitizing recorded action potentials and/or local field potentials;
c) reducing the influence of current dipoles located far away from the electrodes to obtain signals that are decoupled from other anatomical structures;
wherein said action potentials and/or local field potentials (LFPs) further have been subjected to at least one of the following four steps
d1) high-pass filtering of the signals and amplitude thresholding at a predefined value above noise background for the identification of action potentials;
d2) obtaining at least one spike train from trains of action potentials generated from groups of neurons or individual cells;
d3) creating peristimulus time histograms (PSTHs) from spike trains
g) creating evoked potentials (EPs) from the LFPs;
and wherein the feature vectors have been transformed by a coefficient matrix obtained from principal component analysis (PCA) or related methods.

10. A method according to claim 1, wherein each state of the CNS is identified as being one of at least three reference states.

11. The method according to claim 1, wherein the action potentials and/or local field potentials have been obtained from an awake animal or human.

12. The method according to claim 1, wherein the action potentials and/or local field potentials have been obtained from at least one anatomical structure located below the superficial structures of the brain.

13. Use of the method according to claim 1 for assessment of a state of the CNS and/or differentiation of at least two states of the CNS.

14. Use of the method according to claim 1 for evaluating the effect of a treatment of a condition or disease, wherein the condition or disease is neurological and/or psychiatric.

15. Use according to claim 14, wherein the condition is Parkinson's disease.

16. Use according to claim 14, wherein the condition is schizophrenia.

17. Use according to claim 14, wherein the condition is a pain condition.

18. Use according to claim 14, wherein the condition is levodopa-induced dyskinesia.

19. A system for assessment of a state of the central nervous system (CNS) of a subject and/or differentiation between states of the central nervous system (CNS) of a subject at a specific time point based on electrophysiological recordings of signals from at least two anatomical structures, wherein the signals are recorded from recording sites located in the anatomical structures, wherein the electrophysiological recordings comprise spatiotemporal fluctuations in the recorded extracellular potential, wherein the electrophysiological recordings are action potentials and/or local field potentials (LFPs) in the anatomical structures and represent the state of the CNS at said specific timepoint, wherein the system comprises:
means for amplifying recorded action potentials and/or local field potentials;
means for digitizing recorded action potentials and/or local field potentials;
means for reducing the influence of current dipoles located far away from the recording site to obtain signals that are decoupled from other anatomical structures;
said system further comprising at least one of the following four means
means for high-pass filtering of the signals and amplitude thresholding at a predefined value above noise background for the identification of action potentials;
means for obtaining at least one spike train from trains of action potentials generated from groups of neurons or individual cells;
means for obtaining power spectral densities (PSDs) or cross-spectral densities for the LFPs;
means for normalizing PSDs to noise background;
and said system further comprising
means for assessing the state of the CNS based on the recorded spatiotemporal fluctuations defining a location in a predefined state space;
wherein the predefined state space is defined by a set of feature vectors.

20. The system according to claim 19, further comprising:
means for reducing noise and variability by averaging PSDs from different electrode pairs from the same anatomical structure.

21. A system for assessment of a state of the central nervous system (CNS) of a subject and/or differentiation between states of the central nervous system (CNS) of a subject at a specific time point based on electrophysiological recordings of signals from at least two anatomical structures, wherein the signals are recorded from recording sites located in the anatomical structures, wherein the electrophysiological recordings comprise spatiotemporal fluctuations in the recorded extracellular potential, wherein the electrophysiological recordings are action potentials and/or local field potentials (LFPs) in the anatomical structures and represent the state of the CNS at said specific timepoint, wherein the system comprises:
means for amplifying recorded action potentials and/or local field potentials;
means for digitizing recorded action potentials and/or local field potentials;
means for reducing the influence of current dipoles located far away from the recording site to obtain signals that are decoupled from other anatomical structures;
said system further comprising at least one of the following four means
means for high-pass filtering of the signals and amplitude thresholding at a predefined value above noise background for the identification of action potentials;
means for obtaining at least one spike train from trains of action potentials generated from groups of neurons or individual cells;
means for creating peristimulus time histograms (PSTHs) from spike trains;
means for creating evoked potentials (EPs) from the LFPs.
and said system further comprising
means for assessing the state of the CNS based on the recorded spatiotemporal fluctuations defining a location in a predefined state space;
wherein the predefined state space is defined by a set of feature vectors.

22. The system according to claim 21, further comprising
means for providing a stimulus to the CNS.

23. The system according to claim 19, further comprising:
means defining the set of feature vectors of the predefined state space based on features of signals obtained in electrophysiological recordings under at least three reference states.

24. The system according to claim 19, further comprising
means for projecting a feature vector onto a subspace spanned by at least one of the reference states.

25. The system according to claim 19, further comprising
at least three electrodes.

26. The system according to claim 25, wherein each electrode has a diameter up to 100 μm and wherein each electrode is stiff enough to penetrate neuronal tissue and into the anatomical structure.

27. The system according to claim 25, wherein each electrode has a diameter up to 100 μm and wherein at least one electrode is flexible in at least one dimension.

28. The system according to claim 19, further comprising
a recording device being able to be connected to at least three electrodes and having the ability to record action potentials and/or local field potentials in said anatomical structure, wherein the recorded action potentials and/or local field potentials at one specific timepoint represent the state of the CNS at said specific timepoint.

29. Use of the system according to claim 19 for assessment of a state of the CNS and/or differentiation of at least two states of the CNS.

30. Use of a system according to claim 19 for evaluating the effect of a treatment of a condition or disease, wherein the condition or disease is neurological and/or psychiatric.

31. Use according to claim 30, wherein the condition is Parkinson's disease.

32. Use according to claim 30, wherein the condition is schizophrenia.

33. Use according to claim 30, wherein the condition is a pain condition.

34. Use according to claim 30, wherein the condition is levodopa-induced dyskinesia.
